(19) United States
(12) Patent Application Publication
(10) Pub. No.: US 2005/0009790 A1
(43) Pub. Date: Jan. 13, 2005

(54) Ear and wound treatment

(76) Inventor: Laszlo Sichtnik, Riverdale, NY (US)

Correspondence Address: LITMAN LAW OFFICES, LTD., P.O. BOX 15035 CRYSTAL CITY STATION, ARLINGTON, VA, 22215, US

(21) Appl. No.: 10/614,298

(22) Filed: Jul. 8, 2003

(51) Int. Cl. A61K 31/69

(52) U.S. Cl. 514/155

(57) ABSTRACT

The invention is directed to a method of treating otitis media, otitis externa, and wounds to animal and human skin where tissue healing is delayed due to skin maceration caused in part by wound discharge exacerbated by e.g., folds of skin. More specifically, the invention relates to treating chronic otitis externa and media in subjects such as dogs and cats by administering to a subject in need of such treatment an effective amount of a pharmaceutical mixture, in the form of a powder, to mop up discharge, reduce pain, reduce inflammation, and reduce harmful populations of bacteria and fungi, and thereby promote rapid recovery from otitis media and/or otitis externa. The invention is further directed to providing an apparatus to deliver a powder composition to the ear of a subject such as a dog or cat.

### BACKGROUND OF THE INVENTION

1. Field of the Invention

This invention relates to a method of treating otitis media and otitis externa in animals such as dogs and cats. The invention also relates to a method of treating a wound in skin fold dermatitis.

2. Description of the Related Art

Ear infections are very common in dogs, and less so in cats. Two types of ear infection are common: otitis externa, infection of the external ear canal (“outer ear”), and otitis media, infection of the middle ear. Dogs with pendulous ears such as Cocker Spaniels, Standard Poodles, and Cavalier King Charles Spaniels, are particularly vulnerable to outer ear infection.

The causative agents of otitis externa include bacteria and/or yeasts. However, all healthy dogs have bacteria and yeasts in their outer ear and problems generally arise when something untoward happens that causes an unhealthy perturbation in the populations of the bacteria and fungi organisms in the outer ear. Other causes include an accumulation of wax, thick or matted hair in the ear canal, ear mites, a foreign body such as a grass seed, a tumor or impaired drainage of the ear. Sometimes, infections of the external ear canal are a secondary result of some other generalized condition such as allergies. Otitis media usually results from the spread of infection from the external ear canal to the middle ear. Also, foreign bodies, debris, ulceration or improper ear cleaning can rupture the eardrum and allow infection to reach the middle ear.

Chronic otitis media and externa typically results in a congested middle ear and ear canal, respectively. For example, congestion can take the form of an inflammatory response in the mucosal tissue of the middle ear, resulting in fluid effusion in the middle ear, wherein the resulting fluid is viscous and pus-filled. Such fluid, including puss, can cause an unhealthy increase in bacteria and/or yeast populations which in turn leads to further congestion and fluid production thereby providing a continuous moist environment that acts to further increases the populations of bacteria and fungal organisms. For example, ceruminous exudate can predisposes a dog to a yeast infection of the ear. A dog with an infected ear will typically repeatedly scratch the infected ear thereby increasing the risk of ear infection.

Several efforts have been made to address these problems. U.S. Pat. No. 6,423,694 B1, issued Jul. 23, 2002 to Drutz et al., describes a method of promoting drainage of congested middle ear fluid. The method comprises administering to the middle ear of the subject a uridine triphosphate such as uridine 5′-triphosphate (“UTP”), or an analog of UTP, in an amount effective to promote drainage of congested middle ear fluid by hydrating mucous secretions in the middle ear or by stimulating ciliary beat frequency in the middle ear or Eustachian tube.

U.S. Pat. No. 4,331,686, issued May 25, 1982 to S. Djurickovic, describes a method of treating otitis externa in dogs by administering to the infected animal a dosage effective to alleviate the symptoms of the infection of a composition comprising beta-(1-adamantyl)-alpha,alpha-dimethylethylamine or its acid salt. The '686 composition is limited to treating otitis externa in dogs. Thus, there is a need for a composition of sufficient efficacy to treat both otitis media and externa.

To be effective as a treatment of otitis, it is preferable that a medication contacts the infected area, which may be deep in the ear canal. To achieve this kind of contact, the medication must be placed deep within the canal while minimizing the risk of injury to e.g. a dog's ear drum (tympanum). Thus if a syringe or like device is used to place a medication deep into an infected ear, the syringe should be adequate for its intended purpose, namely safe delivery of medication deep into a dog's ear.

U.S. Patent Publication No. 20020139088 A1, published Oct. 3, 2002, shows a syringe having a syringe body of a norbornene and ethylene copolymer, the body defining a chamber for containing water and having an opening, a plunger seal of a halobutyl based elastomer sealing the opening; and wherein the syringe meets all requirements of the United States Pharmocopoeia for sterile water for injection. The Woodworth et al. syringe is not designed to deliver powder based formulations into a dog's ear. In fact, the Woodworth et al. syringe is essentially designed to store or deliver sterile water without contaminating the sterile water with halogens. In addition, Woodworth et al. syringe is not suitable for delivering a powder composition into a dog's ear. For example, the Woodworth et al. syringe is not specifically designed to avoid accidental over insertion of the syringe into a dog's ear thus risking, for example, accidental contact with a dog's tympanum (ear drum). Thus, there is a need for a syringe or similar delivery mechanism for safe delivery of a powder based formulation into an animal's ear such as a dog or cat ear.

Other publications showing medications or devices for treating otitis in animals such as dogs and cats but which do not solve the above mentioned problems include U.S. Pub. No. US 2002/0131903 A1, published Sep. 19, 2002 (device for aspirating and dispensing liquid samples); U.S. Pub. No. US 2002/0141986 A1, published Oct. 3, 2002 (use of antimicrobial proteins and peptides for the treatment of otitis media and paranasal sinusitis); U.S. Pub. No. US 2002/0142999 A1, published Oct. 3, 2002 (antibiotic compositions for treatment of the eye, ear and nose); and U.S. Pub. No. US 2002/0143298 A1, published Oct. 3, 2002 (blunt cannula and filter assembly and method of use with point-of-care testing cartridge).

Other patents showing medications or devices for treating otitis in animals such as dogs and cats but which do not solve the above mentioned problems include U.S. Pat. No. 2,385,262, issued Sep. 18, 1945 to D. Curtis (therapeutic-anesthetic preparations); U.S. Pat. No. 4,025,620, issued May 24, 1977 to Beyer et al. (treatment of canine otitis and compositions therefore); U.S. Pat. No. 4,169,065, issued Sep. 25, 1979 to R. D. Robertson (ear cleaning mixture for canine); U.S. Pat. No. 4,278,664, issued Jul. 14, 1981 to V. Cleave (preventative treatment for otitis externae); U.S. Pat. No. 4,954,334, issued Sep. 4, 1990 to Pugh et al. (foot powder composition); U.S. Pat. No. 5,863,941, issued Jan. 26, 1999 to R. K. Liedtke (method and composition of a topical treatment of inner ear and labyrinth symptoms); and U.S. Pat. No. 6,093,417, issued Jul. 25, 2000 to E. J. Petrus (composition to treat ear disorders).

Further patents showing medications or devices for treating otitis in animals such as dogs and cats but which do not solve the above mentioned problems include U.S. Pat. No. 6,413,499, issued Jul. 2, 2002 to B. M. Clay (methods and kits for maxillary dental anesthesia by means of a nasal deliverable anesthetic); U.S. Pat. No. 6,420,425, issued Jul. 16, 2002 to S. A. Melman (method for the broad based treatment of infections especially infections of organs such as the skin and vagina); PCT Pub. No. WO 92/11016, published 9 Jul. 1992 to FISONS PLC, GB (pharmaceutical composition containing UTP for the treatment of cystic fibrosis); and PCT Pub. No. WO 96/39146 to Bayer Corporation (non-irritation, non-sensitizing, non-ototoxic otic antibacterial compositions).

None of the above inventions and patents, taken either singly or in combination, is seen to describe the instant invention as claimed. Thus a method of treating otitis externa and media solving the aforementioned problems is desired.

### SUMMARY OF THE INVENTION

The invention is directed to a method of treating otitis media, otitis externa, and wounds to animal and human skin where tissue healing is delayed due to skin maceration caused by excessive wound discharge as sometimes occurs in skin fold dermatitis. More specifically, the invention relates to treating chronic otitis externa and media in subjects such as dogs and cats by administering to a subject in need of such treatment an effective amount of a pharmaceutical mixture, in the form of a powder, to mop up discharge, reduce pain, reduce inflammation, and reduce harmful populations of bacteria and fungi, and thereby promote rapid recovery from otitis media and/or otitis externa. The invention is further directed to providing an apparatus to deliver a powder composition to the ear of a subject such as a dog or cat.

Accordingly, it is a principal object of the invention to provide a method of treating otitis externa in a subject in need of such treatment.

It is also an object of the invention to provide a method of treating otitis media in a subject in need of such treatment.

It is another object of the invention to provide a method of treating a skin wound liable to skin maceration such as in skin fold dermatitis in a subject in need of such treatment.

It is yet another object of the invention to provide a powder applicator for delivering a powder based composition to the ear.

It is a further object of the invention is to provide a powder applicator that can deliver a powder based composition to the ear without risk of damaging the ear tympanum.

It is an object of the invention to provide improved elements and arrangements thereof in an apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is an environmental, perspective view of a powder applicator according to the invention.

FIG. 2A is a perspective view of a powder applicator according to the invention wherein a piston is in an up position.

FIG. 2B is a perspective view of the powder applicator of FIG. 2A wherein the piston is in a down position.

FIG. 3A is a perspective view of a powder applicator according to the invention wherein a cotton tip applicator is in an up position.

FIG. 3B is a perspective view of the powder applicator of FIG. 3A with the cotton tip applicator is in a down position.

FIG. 4 is a perspective view of a powder applicator designed particularly to avoid accidental contact with a subject's tympanum.

Similar reference characters denote corresponding features consistently throughout the attached drawings.

### DETAILED DESCRIPTION OF THE PREFERRED EMBODIMENTS

The present invention relates to a method of treating otitis media, otitis externa, and wounds to animal and human skin where tissue healing is delayed due to skin maceration caused by wound discharge. More specifically, the invention relates to treating chronic otitis externa and media in subjects such as dogs and cats by administering to a subject in need of such treatment an effective amount of a pharmaceutical mixture, in the form of a powder, to mop up discharge, reduce pain, reduce inflammation, and reduce harmful populations of bacteria and fungi, and thereby promote rapid recovery from otitis media and/or otitis externa. The invention is further directed to providing an apparatus to deliver a powder composition to the ear of a subject such as a dog or cat.

In one embodiment of the invention a method is provided for treating a subject suffering from otitis media and/or externa, the method comprising the step of administering to a subject in need of such treatment a pharmaceutical mixture, in the form of a powder, comprising of pharmaceutically effective amounts of a local anesthetic agent, an antimicrobial agent, an anti-inflammatory agent, and an integrator.

The pharmaceutical mixture may further comprise an anti-caking agent such as lactose powder to prevent caking of the pharmaceutical mixture and to improve adhesion of the powder composition on the skin surface. The exact form of the local anesthetic is not critical providing that the local anesthetic is provided in powder form such as norcain in the form of crystalline powder. Similarly, the exact form of the antimicrobial agent is not critical providing it is in powder form such as [4-chlorphenyl]-3,4-dichlor-benzol-sulfonamidum powder. Likewise for the anti-inflammatory agent and the integrator agent, which can be boric acid powder and urea powder, respectively. The integrator facilitates the mixing of the other ingredients, i.e. the local anesthetic, antimicrobial, and anti-inflammatory agent, with wax produced by the ears.

It is believed that the pharmaceutical mixture of the invention is the only truly reliable treatment for chronic otitis externa and media in dogs and cats.

The powder composition also produces significantly better results than that achieved by the prior art with regard to treating wounds located in moist areas of a subject's body that are prone to skin fold dermatitis such as under a fold of skin at the junction between the scrotum and the inner thigh. Folds of skin can create an unventilated skin fold that negatively impacts on the effectiveness of current and prior art methods of treating wounds. In contrast, the powder composition of the present invention dries the skin wound out by reducing the discharge production (e.g. by mopping up or reducing the discharge of puss) that helps prevent skin maceration.

The powder composition of the invention also creates a hostile environment for bacteria and fungi in wounds in folded skin. Reduction of bacteria and fungi problems in turn prevents excessive irritation at the site of the wound, which in turn leads to less acts of scratching on the part of the subject (e.g. human, cat, or dog subject) thereby promoting healing of the wound such as a wound under a fold of skin.

In a preferred embodiment of the invention, the pharmaceutical mixture consists essentially of norcain powder, [4-chlorphenyl]-3,4-dichlor-benzol-sulfonamidum, boric acid powder, urea powder, and lactose powder.

### EXPERIMENTAL

The present invention will be explained further with examples devoted to the preferred embodiments of the invention.

### Example 1

### Treatment of Chronic Otitis Media in a Cat

The pharmaceutical composition of the invention was administered to a cat with chronic otitis media. Specifically, prior to treatment with the powder composition of the invention, the cat suffered from a chronic bacterial middle ear infection, including a pus-like discharge, for a considerable period of time. A cultural-sensitivity test was performed and the appropriate antibiotic was administered both as ear drops and orally over a 2 month period, but the chronic otitis media and discharge was not cured.

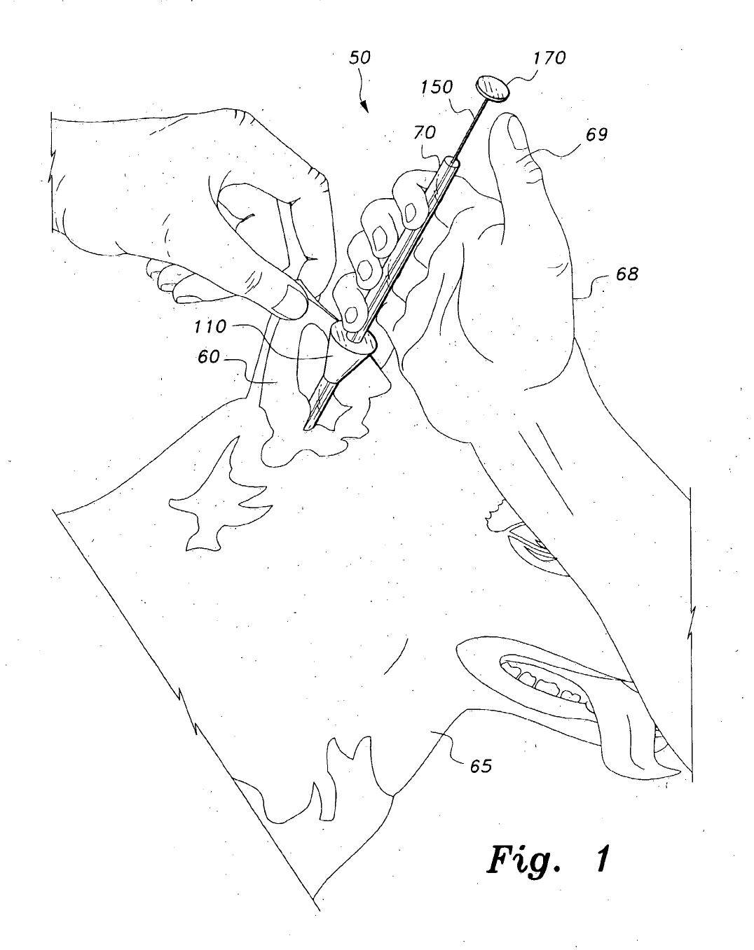

At this point, the cat was treated with 6 treatments of the powdered composition of the invention, specifically a powder composition containing pharmaceutically effective amounts of norcain powder, [4-chlorphenyl]-3,4-dichlor-benzol-sulfonamidum, boric acid powder, urea powder, and lactose powder). More specifically, each treatment comprises of about 0.2 cc to about 0.3 cc of a pharmaceutically mixture made of 3 grams [4-chlorphenyl]-3,4-dichlor-benzol-sulfonamidum, 4 grams of norcain powder, 100 milligrams of carbamid (urea), 4 grams of boric acid powder, and 6 grams of lactose powder. Each treatment was applied using an applicator 50 (see, e.g., FIG. 1). After just 6 treatments over a two-week period the discharge stopped, and the diseased ear dried up and healed.

It is hypothesized that the remarkable healing attributes of the powder composition of the invention is due in part to the ability of the powder composition to provide a high concentration of antimicrobial agent, such as boric acid powder, at the source of the otitis. In contrast to drops or ointment as used in the prior art for treating ear infections, the powder composition acts as an absorbent for sebaceous and ceruminous gland product associated with ear infections.

The powder composition is able to reach the site of the otitis far easier than e.g. an antibiotic administered via the blood stream. Specifically, while the active muscle parts of the body are well served by blood flow (e.g. the main leg muscles), this does not necessarily apply to areas inside the ear which are not well served by blood thus preventing a pharmaceutically effective amount of antibiotic to reach the site of ear infection.

### Example 2

Treatment of a 1 Inch Long and 1/4 Inch Deep Wound

A 1 inch long and 1/4 inch deep wound in a human subject in the vicinity of an unventilated skin fold in the groin area of the subject at the junction between the scrotum and the inner thigh. Prior to treatment with the powdered composition of the invention, the patient felt a burning sensation at the site of the wound with noticeable discharge of puss. Conventional treatment including topical treatment with an antiseptic and wound healing ointment for 1 week resulted in maceration of the skin and wound.

Upon application of the powdered composition of the invention the burning sensation quickly reduced and the discharge decreased significantly. More specifically, each treatment comprises of about 0.2 cc to about 0.3 cc of a mixture made of 3 grams [4-chlorphenyl]-3,4-dichlor-benzol-sulfonamidum, 4 grams of norcain powder, 100 milligrams of carbamid (urea), 4 grams of boric acid powder, and 6 grams of lactose powder. In addition, the size of the wound decreased by 50% and after 4 days of treatment the granulation tissue filled-up the wound and fresh epithelial border started to develop in the reduced wound.

In another embodiment of the invention, an applicator is provided for delivering the pharmaceutical mixture of the invention to the inside of a subject's ear such as the ear of a dog with otitis.

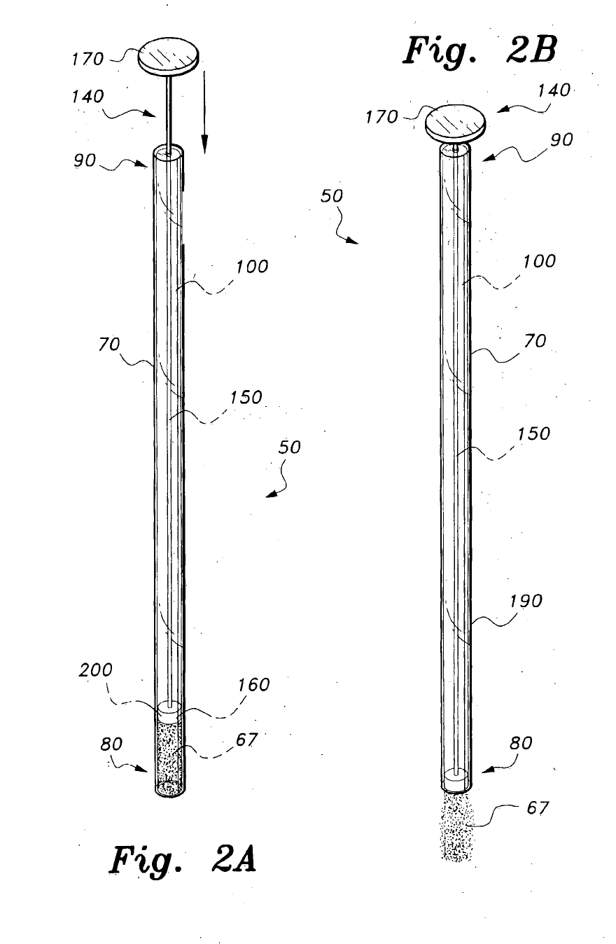

FIG. 1 shows an environmental perspective view of one embodiment of the applicator 50. The applicator 50 is shown partly inserted into a ear 60 of a subject, here shown as a dog 65. A vet is shown administering a powder composition 67 (see e.g. FIG. 2A) into the dog's ear 60. Specifically, a vet's hand 68 is shown positioning the applicator 50 into the dog's ear 60.

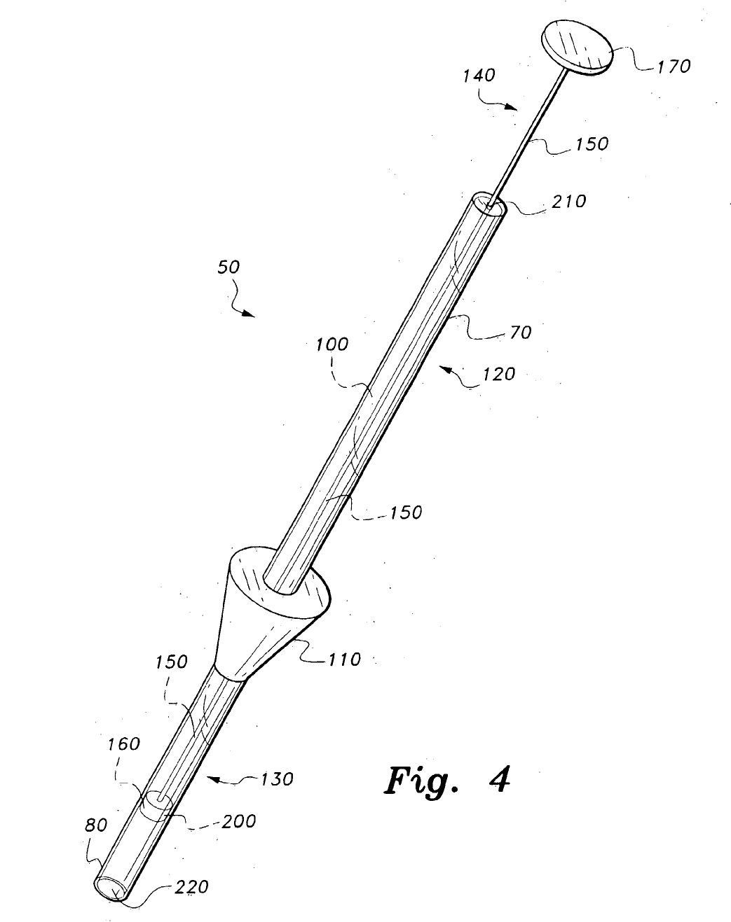

Referring to FIGS. 2A, 2B, 3A, 3B, and 4, the applicator 50 generally comprises a hollow applicator body 70 of generally tubular shape defining a distal 80 and proximal 90 opposite ends. The applicator body 70 further defines a bore 100. An optional ear guard 110 (see FIG. 4) is fitted around the hollow applicator body 70 thereby defining an upper 120 and lower 130 sections of the hollow applicator body 70. The upper section 120 is preferably griped by a vet's hand 68 as shown in FIG. 1.

With respect to FIGS. 2A and 2B, the hollow bore 100 houses a plunger 140 comprising a piston rod 150 with opposite ends defining a piston 160 and a flange or rod handle 180. The piston 160 is sized to fit snugly inside the hollow applicator body 70, and more particularly is sized to just fit inside the bore 100 defined by the sidewall 190 of the hollow applicator body 70. Specifically, the piston 160 defines a piston surface 200 which is in proximate contact with the side wall 190. The piston surface 200 may be made of a low friction coefficient material such as Teflon™ (PTFE, i.e. polytetrafluoroethylene) to facilitate reciprocal movement of the plunger 140 up and down inside the bore 100.

The flange or handle 170 is preferably sized to allow easy contact with a digit of a veterinarian's (“vet's”) hand such as a vet's thumb 69 (see FIG. 1). The proximal end 90 of the hollow applicator 70 defines an orifice 210 sized to just accommodate the width of the plunger rod 150 thereby permitting the plunger rod 150 to just pass through the orifice 210. The piston surface 200 and orifice 210 act in combination to keep the piston rod 150 axially aligned inside the bore 100 without contacting the sidewall 190 of the hollow applicator body 70.

The distal end 80 defines an opening 220 of sufficient size to permit a powder composition 67 to be expelled from the bore 100 and into the ear cavity, and more particularly the ear canal, of a subject such as a dog 65.

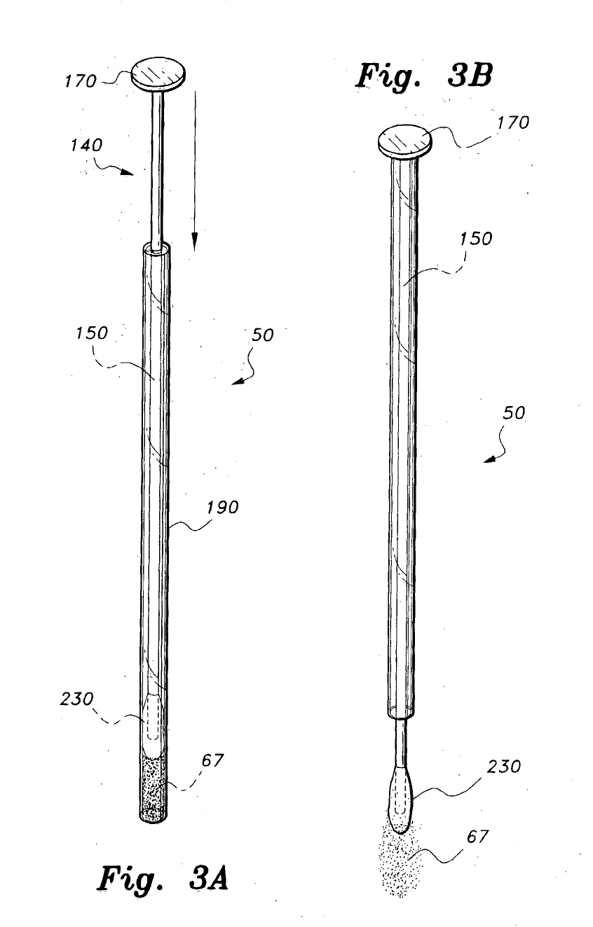

With respect to FIGS. 3A and 3B, the piston 160 is replaced with a cotton tip end 230. Thus, when the plunger 140 is pushed down the cotton tip end 230 both expels the powder composition 67 and can be used by a vet to spread the powder composition 67 in the ear canal of a subject's ear.

With respect to FIG. 4, an optional ear guard 110 is shown fitted around the hollow applicator body 70 thereby defining an upper 120 and lower 130 sections of the hollow applicator body 70. The optional ear guard 110 serves at least two functions. The guard 110 may be positioned on the applicator body 70 to stop the applicator 50 being inserted too deep into the subject's ear and damaging e.g. the ear's tympanum. The guard 110 can also function as a barrier preventing the pharmaceutical composition of the invention spilling out of the ear 60. It should be understood that the optional ear guard 110 can take various forms such as a flap fitted around the hollow applicator body 70.

It should be understood that the term “pharmaceutically effective amount” means an effective amount of the pharmaceutical pharmaceutical composition of this invention can be varied so as to achieve the desired therapeutic response for a particular subject, compositions and mode of administration. The selected dosage level will depend to some extent upon the severity of the otitis condition being treated and the condition and prior medical history of the subject being treated. However, it is within the skill of the art to start doses of the pharmaceutical mixture of the present invention at levels lower than required to achieve the desired therapeutic effect and to gradually increase the dosage until the desired effect is achieved.

It is to be understood that the present invention is not limited to the embodiments described above, but-encompasses any and all embodiments within the scope of the following claims.

1. A method of treating otitis media and otitis externa, the method comprising the step of administering to a subject in need of such treatment a pharmaceutical mixture, in the form of a powder, the powder comprising pharmaceutically effective amounts of a local anaesthetic agent, an antimicrobial agent, an anti-inflammatory agent, and an integrator.

2. The method of claim 1, wherein the pharmaceutical mixture further comprises an anti-caking agent to prevent caking of the pharmaceutical mixture.

3. The method of claim 2, wherein the anti-caking agent is lactose powder.

4. The method of claim 1, wherein the local anaesthetic is norcain powder.

5. The method of claim 1, wherein the antimicrobial agent is [4-chlorphenyl]-3,4-dichlor-benzol-sulfonamidum powder.

6. The method of claim 1, wherein the anti-inflammatory agent is boric acid powder.

7. The method of claim 1, wherein the integrator is urea powder.

8. The method of claim 1, wherein the pharmaceutical mixture consists essentially of norcain powder, [4-chlorphenyl]-3,4-dichlor-benzol-sulfonamidum powder, boric acid powder, urea powder, and lactose powder.

9. A method of treating a wound in skin fold dermatitis in a subject, the method comprising the step of administering to a subject in need of such treatment a pharmaceutical mixture, in the form of a powder, comprising of pharmaceutically effective amounts of a local anaesthetic agent, an antimicrobial agent, an anti-inflammatory agent, and an integrator, wherein the mixture is applied to the region of the wound thereby substantially drying out the wound thereby avoiding formation of macerated skin.

10. The method of claim 9, wherein the pharmaceutical mixture consists essentially of norcain powder, [4-chlorphenyl]-3,4-dichlor-benzol-sulfonamidum powder, boric acid powder, urea powder, and lactose powder.

11. An applicator adapted for administering a pharmaceutical powder composition into a subject's ear, comprising a hollow applicator body adapted to hold a pharmaceutical powder composition, and a plunger adapted to expel the pharmaceutical powder composition from the applicator body into a ear canal thereby providing an applicator adapted for administering the pharmaceutical powder composition into a subject's ear.

12. The applicator of claim 11 further comprising a ear guard to prevent over insertion of the applicator body into the subject's ear thereby providing a substantially safe way of administering the pharmaceutical powder into the subject's ear.

13. The applicator of claim 11, wherein the plunger comprises a cotton tip for both expelling the pharmaceutical powder composition and spreading the composition inside the ear canal.

14. A pharmaceutical composition for treating otitis media and otitis externa, comprising pharmaceutically effective amounts of a local anaesthetic agent, an antimicrobial agent, an anti-inflammatory agent, and an integrator.

15. The pharmaceutical composition of claim 14 further comprising an anti-caking agent to prevent caking of the pharmaceutical mixture.

16. The pharmaceutical composition of claim 15, wherein the anti-caking agent is lactose powder.

17. The pharmaceutical composition of claim 14, wherein the local anaesthetic is norcain powder.

18. The pharmaceutical composition of claim 14, wherein the antimicrobial agent is [4-chlorphenyl]-3,4-dichlor-benzol-sulfonamidum powder.

19. The pharmaceutical composition of claim 14, wherein the anti-inflammatory agent is boric acid powder.

20. The pharmaceutical composition of claim 14, wherein the integrator is urea powder.

21. The pharmaceutical composition of claim 14, wherein the pharmaceutical composition consists essentially of norcain powder, [4-chlorphenyl]-3,4-dichlor-benzol-sulfonamidum, boric acid powder, urea powder, and lactose powder.

22. A pharmaceutical composition for treating a wound in a human subject, comprising pharmaceutically effective amounts of a local anaesthetic agent, an antimicrobial agent, an anti-inflammatory agent, and an integrator.

23. The pharmaceutical composition of claim 22 further comprising an anti-caking agent to prevent caking of the pharmaceutical mixture.

24. The pharmaceutical composition of claim 23, wherein the anti-caking agent is lactose powder.

25. The pharmaceutical composition of claim 22, wherein the local anaesthetic is norcain powder.

26. The pharmaceutical composition of claim 22, wherein the antimicrobial agent is [4-chlorphenyl]-3,4-dichlor-benzol-sulfonamidum powder.

27. The pharmaceutical composition of claim 22, wherein the anti-inflammatory agent is boric acid powder.

28. The pharmaceutical composition of claim 22, wherein the integrator is urea powder.

29. The pharmaceutical composition of claim 22, wherein the pharmaceutical composition consists essentially of norcain powder, [4-chlorphenyl]-3,4-dichlor-benzol-sulfonamidum, boric acid powder, urea powder, and lactose powder.
